(19) United States
(12) Patent Application Publication
(10) Pub. No.: US 2025/0250213 A1
(43) Pub. Date: Aug. 7, 2025

(54) METHOD AND SYSTEM FOR METHANOL SYNTHESIS VIA PLASMA-OXYGEN CARRIER-CATALYSIS COUPLING

(71) Applicant: ZHEJIANG UNIVERSITY, Hangzhou (CN)

(72) Inventors: Hao ZHANG, Hangzhou (CN); Yilin ZHANG, Hangzhou (CN); Kaiyi WANG, Hangzhou (CN); Yanhui LONG, Hangzhou (CN); Jun WANG, Hangzhou (CN); Qunxing HUANG, Hangzhou (CN); Angjian WU, Hangzhou (CN); Xiaodong LI, Hangzhou (CN); Jianhua YAN, Hangzhou (CN)

(21) Appl. No.: 18/822,505

(22) Filed: Sep. 3, 2024

Related U.S. Application Data
(63) Continuation of application No. PCT/CN2024/075380, filed on Feb. 2, 2024.

(30) Foreign Application Priority Data
Dec. 6, 2022 (CN) 202211559277.9

(51) Int. Cl. C07C 1/04

(52) U.S. Cl. CPC C07C 1/043; C07C 2523/825

(57) ABSTRACT

A method and a system for methanol synthesis via plasma-oxygen carrier-catalysis coupling provided. CO₂ is activated and decomposed using an enhanced vibrational-state atmospheric-pressure plasma jet, while H₂O is dissociated by utilizing the heat generated in the plasma environment. An integrated oxygen carrier captures the O₂ produced from the decomposition of CO₂ and H₂O, facilitating forward reactions and enabling the in-situ capture of O₂ from the gas products. This process yields oxygen-free syngas (CO and H₂), which is then efficiently and selectively converted into methanol over a Ni—Ga catalyst at atmospheric pressure. This configuration achieves an orderly conversion of carbon and hydrogen from CO₂ and H₂O into liquid methanol under atmosphere pressure, characterized by high reactant conversion and energy efficiency. Additionally, this method and system support the use of intermittent and distributed renewable energy sources due to their fast on-off capability, high reaction rate, and simple design features.

### TECHNICAL FIELD

The present disclosure pertains to the field of greenhouse gas resource utilization, and specifically relates to a method and a system for methanol synthesis by plasma-oxygen carrier-catalysis coupling.

### BACKGROUND

Carbon emission reduction can be achieved by reducing the utilization rate of fossil fuel energy and improving the efficiency its utilization. In addition, the capture and utilization of carbon dioxide (CO₂) has garnered significant worldwide attention as a method for carbon emission reduction. By capturing, transforming, and repurposing CO₂, substantial amounts can be converted into industrial raw materials and fuels, thereby achieving dual benefits of reducing emissions and optimizing resource utilization.

The technical route of co-converting CO₂ and H₂O to generate low-carbon methanol (CH₃OH) has recently gained considerable traction. CO₂, a prevalent greenhouse gas, serves as a rich carbon source, while H₂O is the most accessible renewable hydrogen source on Earth. Methanol, a liquid product, can leverage existing transportation and power infrastructure, boasting high energy density and clean, efficient combustion—making it an ideal green fuel or hydrogen carrier. Furthermore, methanol ranks as the fourth most significant basic chemical feedstock, capable of producing numerous chemical products. In 2018, four academicians of the Chinese Academy of Engineering (CAE), C. F. Shi, T. Zhang, J. H. Li, and C. L. Bai introduced the strategic concept of “Liquid Sunshine” in Joule, advocating for the conversion of CO₂ and H₂O into green methanol utilizing solar energy. The need for foundational research and development of key technologies for green methanal synthesis is urgent.

However, the conversion process of CO₂ and H₂O into methanol faces several challenges: CO₂ and H₂O exhibit high chemical stability, requiring significant energy for bond dissociation; their decomposition products, CO and H₂, are highly reactive and prone to recombine into CO₂ and H₂O; and the separation of the by-product O₂ adds complexity and cost to the system. Current technologies for CO₂ and H₂O activation and decomposition struggle to address these issues simultaneously.

Therefore, the development of a method and system that can concurrently overcome these challenges—enabling the conversion of CO₂ and H₂O to methanol with minimal carbon and hydrogen loss, under ambient pressure, and in an orderly manner—would significantly enhance the industrial feasibility of carbon utilization. This advancement would present a novel, revolutionary approach to achieving peak carbon dioxide emissions and carbon neutrality.

### SUMMARY

In view of the shortcomings of the prior art, the present disclosure aims to provide a method and a system for methanol synthesis by plasma-oxygen carrier-catalysis coupling. The objective of the present disclosure is achieved through the following technical solutions:

According to a first aspect of this specification, provided is a method for methanol synthesis by plasma-oxygen carrier-catalysis coupling, which includes the following steps:
CO₂ decomposition: activating CO₂ using an enhanced vibrational-state atmospheric-pressure plasma jet, so that the CO₂ is decomposed into O₂ and CO;
H₂O decomposition: decomposing H₂O into O₂ and H₂ by the high temperature generated in the plasma working environment;
Capturing O₂ by an oxygen carrier: using a high-temperature oxygen carrier to absorb the decomposition product O₂ in a CO₂ decomposition reaction zone and a H₂O decomposition reaction zone respectively, so as to separate the O₂ from the decomposition product and obtain oxygen-free CO and H₂ respectively;
Synthesis of methanol: using a Ni—Ga catalyst to facilitate the selective synthesis of methanol from oxygen-free CO and H₂ at atmospheric pressure.

Further, in the step of CO₂ decomposition, a temperature of the enhanced vibrational-state atmospheric-pressure plasma jet is 800-1300° C.

Further, in the step of CO₂ decomposition, the plasma is cooled by H₂O during the H₂O decomposition step.

Further, in the step of O₂ capture by the oxygen carrier, the suitable working temperature of the oxygen carrier is within the plasma temperature range of 800-1300° C.

Further, in the step of O₂ capture by the oxygen carrier, the oxygen carrier is a cerium-perovskite composite oxygen carrier prepared by the sol-gel method.

Further, in the step of methanol synthesis, the Ni—Ga catalyst is prepared by an incipient wetness impregnation method.

According to a second aspect of this specification, provided is a system for methanol synthesis by plasma-oxygen carrier-catalysis coupling, wherein the main body of the system is an enhanced vibrational-state plasma jet reaction device;

The lower part of the enhanced vibrational-state plasma jet reaction device is provided with a plasma jet formation zone formed by an external electrode, an internal electrode, a base and a CO₂ gas flow inlet; a middle part is a two-layer sleeve structure, a space between inner and outer walls forming an oxygen carrier H₂O decomposition reaction zone, and an inner space of the inner wall is communicated with the plasma jet formation zone, forming a plasma-oxygen carrier-water cooled CO₂ decomposition reaction zone;

The external electrode is located at the lower part of the reaction device, has a sleeve-type hollow structure, and is fixed to the base; the inner electrode has a conical structure, is arranged at a lower-middle position within the hollow structure of the outer electrode and is integrally formed by a lower cylinder and an upper frustum. The bottom of the inner electrode is fixed to the base; an upper-middle part of the hollow structure of the external electrode has a structure of a tapered outlet; the CO₂ gas flow inlet is located at the bottom of the reaction device, and CO₂ gas flow enters tangentially from the bottom of the reaction device through the gas flow inlets to form a rotating gas flow inside, which drives an arc between the electrodes to rotate and rise. and the arc is then ejected in a form of plasma jet through the tapered outlet;

An H₂O inlet is positioned below the outer wall of the middle part of the reaction device, and through which H₂O is introduced into the oxygen carrier reaction zone, absorbs the heat provided by the plasma jet in the inner wall, and is decomposed by the oxygen carrier to output oxygen-free H₂; the plasma-oxygen carrier-water cooled CO₂ decomposition reaction zone outputs oxygen-free CO;

The output gases of the two parts of the middle sleeve are mixed at a top of the reaction device, and a CO hydrogenation methanol synthesis reaction zone and a methanol outlet are provided; oxygen-free CO and H₂ are directionally synthesized into methanol at atmospheric pressure over Ni—Ga catalysis, and methanol exits the reaction device through the methanol outlet.

Further, the external electrode and the internal electrode are connected to a frequency-adjustable high-voltage AC power supply, with an adjustable frequency range of 5-40 kHz, a maximum output voltage of 20 kV and a maximum power of 1 kW.

Further, the system includes a CO₂ supply system comprising a CO₂ gas bottle, a mass flow controller and a CO₂ gas valve, wherein the CO₂ gas bottle is used for storing CO₂, the mass flow controller is used for controlling the flow of CO₂ gas, and the CO₂ gas valve is connected with the CO₂ gas flow inlet.

Compared with the background technology, the present disclosure has the following beneficial effects:

(1) The enhanced vibrational-state plasma jet has high electron energy to promote the reaction, while maintaining a low macro gas temperature, resulting in low heat dissipation and high energy efficiency. The high-energy electrons and reactive species in plasma are the main factors to promote the chemical reactions, enabling the reaction to overcome the kinetic barrier of thermochemical reactions and allowing reactions that are difficult to carry out under normal conditions.

(2) The plasma jet effectively activates and decomposes CO₂, achieving both a high conversion rate and energy efficiency. The treatment flow exceeds 5 L·min⁻¹ in a single lab-scale reactor, which can be operated at an atmospheric pressure and is beneficial to application.

(3) The three-dimensional plasma jet reaction zone generated by the enhanced vibrational-state plasma jet is large and separate from the discharge generation zone, so that the discharge stability and the downstream reaction zone do not interfere with each other, promoting the stable operation of the reaction device.

(4) The plasma jet and the enhanced vibrational-state plasma jet are coupled, and the in-situ capture and separation of the decomposition product O₂ are realized by using the strong oxygen binding ability of the oxygen carrier at high temperatures, and the reverse reaction is inhibited at the same time, which significantly improves the conversion rate of CO₂.

(5) The coupled water cooling selectively reduces the temperature of plasma gas, and at the same time, the heated water is efficiently decomposed by oxygen carrier to obtain oxygen-free H₂, which improves the overall energy utilization rate of the system.

(6) The cascade efficient utilization of the plasma energy source is realized, leading to a high reactant conversion rate. The plasma energy source can operate under atmospheric pressure, which is convenient for practical applications.

(7) Quick start and stop, fast reaction rate, and the possible direct use of intermittent and regional renewable energy realize a miniaturized and distributed green methanol supply system based on “zero-carbon power” tailored to local conditions.

### BRIEF DESCRIPTION OF DRAWINGS

FIG. 1 is a schematic diagram of the steps of a method for methanol synthesis by plasma-oxygen carrier-catalysis coupling;

FIG. 2 is a schematic diagram of a system for methanol synthesis by plasma-oxygen carrier-catalysis coupling.

### DESCRIPTION OF EMBODIMENTS

The present disclosure will be further described in detail with the attached drawings and specific embodiments.

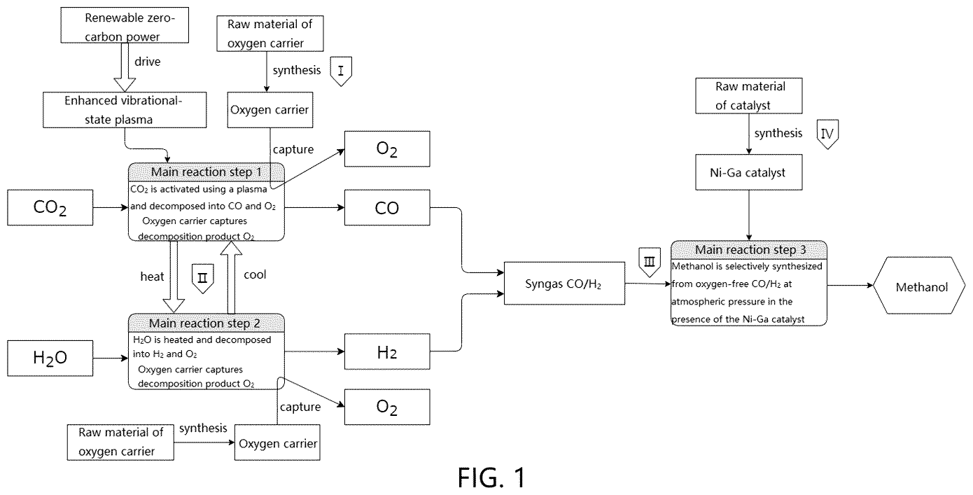

As shown in FIG. 1, the present disclosure provides a method for methanol synthesis by plasma-oxygen carrier-catalysis coupling, which includes the following steps:
main reaction step 1—CO₂ decomposition: activating CO₂ using an enhanced vibrational-state atmospheric-pressure plasma jet, so that the CO₂ is decomposed into O₂ and CO;
main reaction step 2—H₂O decomposition: decomposing H₂O into O₂ and H₂ by the high temperature generated in the plasma working environment;
capturing O₂ by an oxygen carrier: using a high-temperature oxygen carrier to absorb the decomposition product O₂ in a CO₂ decomposition reaction zone and a H₂O decomposition reaction zone respectively, so as to separate the O₂ from the decomposition product and obtain oxygen-free CO and H₂ respectively;
main reaction step 3—synthesis of methanol: using a Ni—Ga catalyst to facilitate the selective synthesis of methanol from oxygen-free CO and H₂ at atmospheric pressure.

In the main reaction step 1, the reaction temperature is 800-1300° C., which is within the temperature of the plasma jet; by optimizing the electrode structure and electrical parameters, the reduced electric field is adjusted and controlled to enhance the vibrational-state level of the plasma jet. The electron energy distribution and vibrational/rotational-state energy level of plasma are determined by in-situ optical emission spectroscopy (OES) diagnosis, the spatial distribution of the temperature field of the plasma jet is measured by a thermocouple, and the plasma discharge characteristics, jet morphology and jet propulsion mechanism are determined by electrical signals and optical signals. By obtaining the above plasma parameters and characteristics, the enhanced vibrational state plasma in the reaction process is effectively regulated.

In the main reaction step 1, the specific detection methods of plasma parameters and characteristics mentioned in the previous paragraph are as follows: using a monochromator equipped with ICCD to obtain the spectrum of the discharge process, and then calculating the electron density according to the Stark broadening method, calculating the electron excitation temperature and vibration temperature according to the Boltzmann curve slope method, and calculating the rotation temperature according to the rotation line fitting method; using an oscilloscope to study the characteristics of discharge electrical parameters, and analyzing the pulse characteristics such as volt-ampere characteristics, pulse frequency and pulse amplitude, and calculating the characteristic parameters such as arc power, conductivity and electric field intensity by combining the obtained temperature and electrical parameters, calculating the reduced electric field (E/N); obtaining the spatial distribution of the axial temperature field of the plasma jet by using mobile thermocouple; recording the motion images of arc and jet by high-speed camera, and obtaining the arc motion characteristics and jet propulsion mechanism.

In the main reaction step 1, CO₂ is decomposed based on the cascade vibration excitation path, which generates low and high-energy vibrational excited molecules of CO₂*(¹Σ⁺) and CO₂*(B₂) in turn through the initial electron collision excitation and subsequent “VV relaxation” processes, among which CO₂*(³B₂) is highly reactive and easily decomposed under the collision of electrons or other particles. This path only needs 5.5 eV energy, which avoids energy waste and has high decomposition efficiency.

In the main reaction step 1, the spatial dynamic distribution of atomic concentrations of CO, CO₂, O₂ and O along the axial direction of the jet is detected based on in-situ molecular beam mass spectrometry and OES, respectively. In the plasma jet-carrier coupling system, the reaction effect, the concentration of the product O₂ and oxygen capture rate of the oxygen carrier are controlled by controlling the spatial position of the carrier, interface temperature, reaction time, gas flow rate and airspeed.

In the main reaction step 1, the specific detection means of the parameters and characteristics of the gas/oxygen carrier mentioned in the previous paragraph are as follows: a two-stage differential pumping system is adopted to generate a sampling molecular beam, so that high-activity chemical components can be “frozen” in situ by entering an ultra-low pressure environment, and then quantitative detection is carried out by quadrupole mass spectrometry equipped with electron ionization to obtain the spatial dynamic distribution of the concentrations of CO, CO₂ and O₂ along the axial direction of the jet; the characteristic spectral lines of O atoms at different axial positions are collected by emission spectroscopy system, and the axial spatial distribution of the O atom density is obtained by combining the intensity of atomic spectral lines and spectral parameters of components with known concentrations.

At the label I, the oxygen carrier used is a cerium-perovskite (LaFeO_{3-δ}) composite oxygen carrier prepared by a sol-gel method. The preparation process is as follows: Ce(NO₃)₃, La(NO₃)₃ and Fe(NO₃)₃ hydrates were dissolved in deionized water to prepare a 0.25 mol/L solution, which was stirred in a water bath at 30° C. for 30 min, and citric acid was added, wherein the citric acid/cation (mol) ratio was 3/1; then the mixture was stirred in a water bath at 50° C. for 30 min to form a chelate, ethylene glycol was added, wherein the ratio of ethylene glycol to cation (mol) was 2/1; then the mixture was stirred in a water bath at 80° C. for 2 hours to form a gel, which was dried at 110° C. for 24 hours, ground into powder, roasted at 400° C. for 4 hours, and then roasted at 900° C. for 6 hours to prepare CeO₂—LaFeO₃ cerium-based perovskite composite material, followed by granulation and reduction to complete the preparation.

At the label II, the water cooling intensity is adjusted by controlling the water flow rate, so as to realize the selective adjustment of the plasma jet temperature in the main reaction step 1 and weaken the reverse reaction of CO₂ decomposition, thus realizing the adjustment of CO₂ decomposition effect; at the same time, the temperature of water heated by heat exchange is controlled by electric auxiliary heating in the main reaction step 2, and oxygen-free H₂ is obtained by efficient decomposition of oxygen carrier.

At the label III, the flow rates of CO and H₂ are controlled respectively, and the high temperature is kept after the main reaction step 3 to prevent liquid products such as methanol from condensing. Then, some product gases can be extracted and analyzed quantitatively in an online gas chromatograph, and indicators such as reactant conversion rate, methanol selectivity and by-product generation amount can be obtained. These indicators can be used to adjust system parameters such as catalyst type, reactant flow rate and ratio, reaction speed and space velocity, so as to optimize whole system.

At the label IV, the Ni—Ga catalysts used can be divided into two types according to the different carriers used. The first method uses ZrO₂ or CeO₂ as the carrier, and its preparation method is as follows: taking a certain amount of ZrO(NO₃)₂·5H₂O and Ce(NO₃)₃·6H₂O, respectively, adding deionized water and stirring until they were completely dissolved, dripping a NH₄OH solution, stirring, filtering, uniformly dispersing the obtained solid in a NH₄OH solution again, then drying at 70° C. for 24 h and calcining at 500° C. for 4 h to obtain CeO₂ or ZrO₂ carrier. A certain amount of a mixed solution of nickel nitrate and gallium nitrate was impregnated on the carrier with a high specific surface area, dried in air at 100° C. for 24 h, and reduced in high purity hydrogen at 700° C. for 2 h. The second method uses SiO₂ as the carrier, and its preparation method is as follows: a certain amount of hydrate of nickel nitrate and gallium nitrate was dissolved in deionized water to obtain a mixed solution, which is then incipient-wetness impregnated on the carrier with a high specific surface area, dried in the air atmosphere of 100° C. for 24 h, and reduced for 2 h in a high-purity hydrogen flow of 700° C.

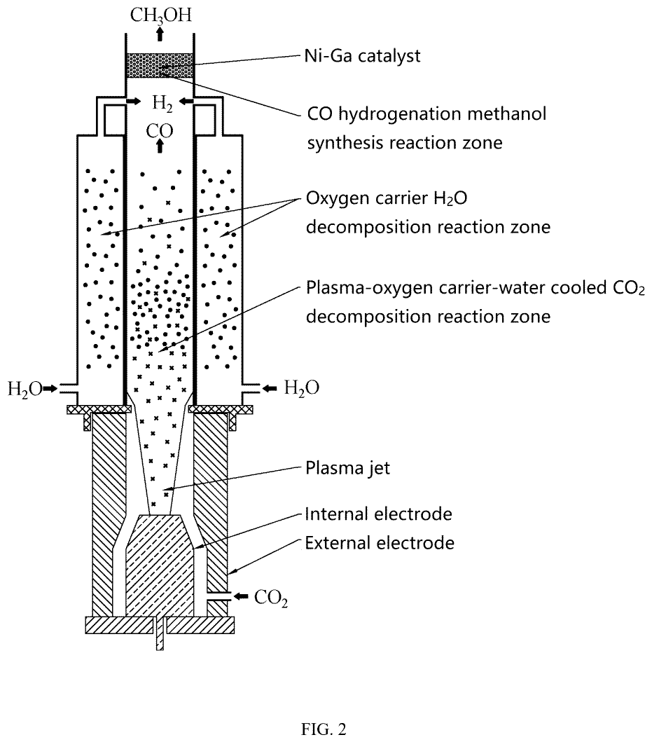

As shown in FIG. 2, the embodiment of the present disclosure provides a system for synthesizing methanol by plasma-oxygen carrier-catalysis coupling, and the main part of the system is a plasma jet reaction device;

A lower part of the enhanced vibrational-state plasma jet reaction device is provided with a plasma jet formation zone formed by an external electrode, an internal electrode, a base and a CO₂ gas flow inlet; a middle part is a two-layer sleeve structure, a space between inner and outer walls forms an oxygen carrier H₂O decomposition reaction zone, and an inner space of the inner wall is communicated with the plasma jet formation zone to form a plasma-oxygen carrier-water cooled CO₂ decomposition reaction zone;

The external electrode is located at the lower part of the reaction device, has a sleeve-type hollow structure and is fixed on the base; the inner electrode has a conical structure, is arranged at a lower-middle position in the hollow structure of the outer electrode and is integrally formed by a lower cylinder and an upper frustum, and the bottom of the inner electrode is fixed on the base; an upper-middle position of the hollow structure of the external electrode has a structure of a tapered outlet; the external electrode and the internal electrode are connected to a frequency-adjustable high-voltage AC power supply, and the power supply has an adjustable frequency of 5-40 kHz, a maximum output voltage of 20 kV and a maximum power of 1 kW; the CO₂ gas flow inlet is positioned at the bottom of the reaction device, and CO₂ gas flow enters tangentially from the bottom of the device through the CO₂ gas flow inlet to form a rotating gas flow inside, which drives an arc between the electrodes to rotate and rise, and the arc is ejected in a form of plasma jet under the action of the tapered outlet;

An H₂O inlet is arranged below the outer wall of the middle part of the reaction device, and through which H₂O is introduced into the oxygen carrier H₂O decomposition reaction zone, absorbs the heat provided by the plasma jet in the inner wall, and is decomposed by the oxygen carrier to output oxygen-free H₂; the plasma-oxygen carrier-water cooled CO₂ decomposition reaction zone outputs oxygen-free CO;

The output gases of the two parts of the middle sleeve are mixed at a top of the reaction device, and a CO hydrogenation methanol synthesis reaction zone and a methanol outlet are provided; oxygen-free CO and H₂ are selectively synthesized into methanol at atmospheric pressure over Ni—Ga catalyst, and the methanol passes out of the reaction device through the methanol outlet.

Further, a CO₂ supply system may be provided, and the CO₂ supply system includes a CO₂ gas bottle, a mass flow controller and a CO₂ gas valve, wherein the CO₂ gas bottle is used for storing CO₂, the mass flow controller is used for controlling the flow of CO₂ gas, and the CO₂ gas valve is connected with the CO₂ gas flow inlet.

The above is only the preferred embodiment of the present disclosure, and although the present disclosure has been disclosed in the above with preferred embodiments, it is not intended to limit the present disclosure. Any person familiar with the field can make many possible changes and modifications to the technical solution of the present disclosure by using the methods and technical contents disclosed above, or modify it into equivalent embodiments with equivalent changes without departing from the scope of the technical solution of the present disclosure. Therefore, any simple modification, equivalent change and modification made to the above embodiment according to the technical essence of the present disclosure without departing from the content of the technical solution of the present disclosure still fall within the scope of protection of the technical solution of the present disclosure.

What is claimed is:

1. A method for methanol synthesis by plasma-oxygen carrier-catalysis coupling, comprising:
CO₂ decomposition: activating CO₂ using an enhanced vibrational-state atmospheric-pressure plasma jet, so that the CO₂ is decomposed into O₂ and CO;
H₂O decomposition: decomposing H₂O into O₂ and H₂ by the high temperature generated in the plasma working environment;
Capturing O₂ by an oxygen carrier: using a high-temperature oxygen carrier to absorb the decomposition product O₂ in a CO₂ decomposition reaction zone and a H₂O decomposition reaction zone respectively, so as to separate the O₂ from the decomposition product and obtain oxygen-free CO and H₂ respectively;
Synthesis of methanol: using a Ni—Ga catalyst to facilitate the selective synthesis of methanol from oxygen-free CO and H₂ at atmospheric pressure.

2. The method for methanol synthesis by plasma-oxygen carrier-catalysis coupling according to claim 1, wherein in the step of CO₂ decomposition, a temperature of the plasma jet is 800-1300° C.

3. The method for methanol synthesis by plasma-oxygen carrier-catalysis coupling according to claim 1, wherein in the step of CO₂ decomposition, the plasma is cooled by H₂O during the H₂O decomposition step.

4. The method for methanol synthesis by plasma-oxygen carrier-catalysis coupling according to claim 1, wherein in the step of O₂ capture by the oxygen carrier, a suitable working temperature of the oxygen carrier is within the plasma jet temperature range of 800-1300° C.

5. The method for methanol synthesis by plasma-oxygen carrier-catalysis coupling according to claim 1, wherein in the step of O₂ capture by the oxygen carrier, the oxygen carrier is a cerium-perovskite composite oxygen carrier prepared by a sol-gel method.

6. The method for methanol synthesis by plasma-oxygen carrier-catalysis coupling according to claim 1, wherein in the step of synthesis of methanol, the Ni—Ga catalyst is prepared by an incipient wetness impregnation method.

7. A system for methanol synthesis by plasma-oxygen carrier-catalysis coupling for implementing the method of claim 1, wherein a main part of the system is an enhanced vibrational-state plasma jet reaction device;
A lower part of the enhanced vibrational-state plasma jet reaction device is provided with a plasma jet formation zone formed by an external electrode, an internal electrode, a base, and a CO₂ gas flow inlet; a middle part is a two-layer sleeve structure, a space between inner wall and outer wall forms an oxygen carrier H₂O decomposition reaction zone, and an inner space of the inner wall is communicated with the plasma jet formation zone to form a plasma-oxygen carrier-water cooled CO₂ decomposition reaction zone;
The external electrode is located at the lower part of the reaction device, has a sleeve-type hollow structure and is fixed on the base; the inner electrode has a conical structure, is arranged at a lower-middle position in the hollow structure of the outer electrode and is integrally formed by a lower cylinder and an upper frustum, and bottom of the inner electrode is fixed on the base; an upper-middle position of the hollow structure of the external electrode has a structure of a tapered outlet; the CO₂ gas flow inlet is arranged at bottom of the reaction device, and CO₂ gas flow enters tangentially from the bottom of the reaction device through the CO₂ gas flow inlet to form a rotating gas flow inside, which drives an arc between the electrodes to rotate and rise, and is ejected in a form of plasma jet under the action of the tapered outlet;
An H₂O inlet is arranged below the outer wall of the middle part of the reaction device, and through which H₂O is introduced into the oxygen carrier H₂O decomposition reaction zone, absorbs heat provided by the plasma jet in the inner wall, and is decomposed by the oxygen carrier to output oxygen-free H₂; the plasma-oxygen carrier-water cooled CO₂ decomposition reaction zone outputs oxygen-free CO;
Output gases of two parts of the middle sleeve are mixed at a top of the reaction device, and a CO hydrogenation methanol synthesis reaction zone and a methanol outlet are provided; oxygen-free CO and H₂ are selectively synthesized into methanol at atmospheric pressure over Ni—Ga catalyst, and the methanol passes out of the reaction device through the methanol outlet.

8. The system according to claim 7, wherein the external electrode and the internal electrode are connected to a frequency-adjustable high-voltage AC power supply, and the power supply has an adjustable frequency of 5-40 kHz, a maximum output voltage of 20 kV and a maximum power of 1 kW.

9. The system according to claim 7, further comprising a CO₂ supply system, comprising a CO₂ gas bottle, a mass flow controller, and a CO₂ gas valve, wherein the CO₂ gas bottle is used for storing CO₂, the mass flow controller is used for controlling flow of CO₂ gas, and the CO₂ gas valve is connected with the CO₂ gas flow inlet.
